(19) United States
(12) Patent Application Publication
(10) Pub. No.: US 2025/0163366 A1
(43) Pub. Date: May 22, 2025

(54) LIGILACTOBACILLUS ANIMALIS STRAIN AND USE THEREOF

(71) Applicant: HEALTH AND HAPPINESS (H&H) HONG KONG LIMITED, Hong Kong (CN)

(72) Inventors: Hua ZHANG, Hong Kong (CN); Lingling ZHAO, Hong Kong (CN); Ying LI, Hong Kong (CN)

(73) Assignee: HEALTH AND HAPPINESS (H&H) HONG KONG LIMITED, Hong Kong (CN)

(21) Appl. No.: 19/028,457

(22) Filed: Jan. 17, 2025

Related U.S. Application Data
(63) Continuation of application No. PCT/CN2023/128860, filed on Oct. 31, 2023.

(30) Foreign Application Priority Data
Oct. 13, 2023 (CN) 202311329687.9

(51) Int. Cl. C12N 1/20; A61K 35/00; A61K 35/744; C12R 1/01

(52) U.S. Cl. CPC C12N 1/205; A61K 35/744; A61K 2035/115; C12R 2001/01

(57) ABSTRACT

A Ligilactobacillus animalis strain, which relates to the field of microbiotechnology. The L. animalis strain has been deposited in Guangdong Microbial Culture Collection Center on Oct. 13, 2022 at GDMCC NO.62867. The L. animalis strain HHP002 can inhibit the expression of cytokines mRNA in the human colon cancer cells, therefor inhibiting the generation of pro-inflammatory cytokines, and then inhibiting the further development of tumors, and can be applied to drugs for preventing and treating human colon cancer.

### CROSS-REFERENCE TO RELATED APPLICATIONS

The present application is a Bypass Continuation Application of PCT Patent Application No. PCT/CN2023/128860, filed on Oct. 31, 2023, which claims priority to Chinese Patent Application No. 202311329687.9, filed on Oct. 13, 2023. The content of all aforesaid applications is incorporated herein by reference.

### FIELD

The present disclosure relates to the technical field of microbiotechnology, in particular to a Ligilactobacillus animalis strain and use thereof.

### BACKGROUND

There are many microorganisms living in human intestines, which not only participate in digestion of food, but also affect people's health.

Nowadays, more and more studies have found that intestinal microorganisms can even affect the immune system. For example, studies have shown that, after Fecal Microbiota Transplantation (FBT), the quantity and function of intestinal T cells in a septic mice have been restored to a certain extent and a length of colon has been obviously restored, while histopathological results show that inflammation and injury of small intestine and colon have been obviously alleviated. During a development of inflammation, cells produce various cytokines, which not only induce proliferation and aggregation of immune cells, but also promote development of tumor cells.

Intestinal cancer is one of the most frequent cancers. At present, types of drug therapy for cancer are relatively simple, mainly including targeted drug treatment and chemotherapeutic drug treatment. The targeted drugs are expensive, and only suitable for patients with specific targets, and there are few suitable people. The chemotherapeutic drugs are low in cost, but side effects are great, which will seriously reduce people's immunity.

Intestinal microorganisms are innate microorganisms in the human intestine, including a large quantity of beneficial probiotics. The production cost of intestinal microorganisms is low, and some intestinal microorganisms can affect people's immune regulation. If the intestinal microorganisms can be used to treat intestinal cancer, it is expected to further improve treatment quality and reduce treatment cost, but there is no relevant report yet in prior art.

### SUMMARY

In view of the shortcomings of the prior art, the present disclosure aims to provide a Ligilactobacillus animalis strain to improve treatment quality for intestinal cancer and reduce treatment cost.

To achieve the purpose, schemes of the present disclosure are as follows.

A Ligilactobacillus animalis strain, which has been deposited in the Guangdong Microbial Culture Collection Center (GDMCC) on Oct. 13, 2022 with deposit number GDMCC NO.62867.

A use of the L. animalis strain in a preparation of a pharmaceutical composition for inhibiting an expression of pro-inflammatory cytokine in human colon cancer.

The use of the L. animalis strain in the preparation of a pharmaceutical composition for inhibiting an expression of pro-inflammatory cytokines in human colon cancer, the pro-inflammatory cytokine is one or more of IL-1B, IL-6, IL-8, and TNF-α.

A use of the L. animalis strain in a preparation of a pharmaceutical composition for treatment of human colon cancer.

A use of the L. animalis strain in a preparation of a pharmaceutical composition for prevention of human colon cancer.

A use of the L. animalis strain in a preparation of a probiotic formulation.

Benificial effects: the present disclosure provides a L. animalis strain with deposit number GDMCC NO.62867. After acting on human colon cancer cells, the L. animalis strain can obviously inhibit an up-regulation of pro-inflammatory cytokines IL-1β, TNF-α, IL-6 and IL-8 in human colon cancer cells, and can be applied to drugs for preventing and treating human colon cancer.

### BRIEF DESCRIPTION OF THE DRAWINGS

FIG. 1 is the results of gene expression levels of IL-1β, IL-6, IL-8, IL-17, and TNF-α in HT29 cells from the cell experiment of Embodiment 1.

FIG. 2 is the result of acid resistance experiments of the L. animalis strain HHP002.

FIG. 3 is the result of choline resistance experiments of the L. animalis strain HHP002.

FIG. 4 is the cytotoxicity test result of the L. animalis strain HHP002.

### DETAILED DESCRIPTION OF EMBODIMENTS

The present disclosure provides a Ligilactobacillus animalis strain and use thereof. In order to make the purposes, technical schemes, and effects of the present disclosure more clear and definite, the present disclosure is further described in detailed below with reference to the embodiment and the attached drawings. It should be understood that the embodiments described herein are only used to explain the present disclosure, not to limit the present disclosure.

In the below embodiments, the L. animalis strain HHP002 has been deposited in the Guangdong Microbial Culture Collection Center (GDMCC) on Oct. 13, 2022, and the deposit number of the L. animalis strain A-11-15 is GDMCC NO.62867.

### Embodiment 1, Cell Experiments

1. Human colon cancer cells HT29 were cultured.

The human colon cancer cells HT29 were incubated with RPMI1640 (HyClone, UT, USA) medium in 5% CO₂ humidified at 37° C., and the medium contained 10% of FBS (Gibco, Grand Island, NE, USA) and 1% of antibiotics (100 mg/mL of streptomycin and 100 U/mL of penicillin). The HT29 cells were seeded in a 12-well cell culture plate at a density of 5×10⁵ cells per well.
2. The Human colon cancer cells HT29 and the L. animalis strains HHP002 were co-cultured.
2.1 Four groups were set, including a control group, a HHP002 group, a LPS group, and a LPS+HHP002 group.
2.2 The process acting on the control group was as follows: the HT29 cells were cultured and without other treatment;

The process acting on the HHP002 group was as follows: when a confluence of the HT29 cells reached 80%, the HT29 cells were put into a cell culture medium containing L. animalis strain HHP002 (a concentration of the L. animalis strain was 1×10⁷ CFU/mL) for co-culturing for 6 hours;

The process acting on the LPS group was as follows: when a confluence of the HT29 cells reached 80%, 10 μg/mL of LPS was added, treating for 12 hours;

The process acting on the LPS+HHP002 group was as follows: when a confluence of HT29 cells reached 80%, the HT29 cells were first put into in a cell culture medium containing L. animalis strain HHP002 (a concentration of the L. animalis strain was 1×10⁷ CFU/mL) for co-culturing for 6 hours, and then 10 μg/mL of LPS was added, treating for 12 hours.
2.3 After treatments, each group was washed with PBS for 3 times, and then the HT29 cells were lysed with RNAiso170Plus (Takara, Dalian, China) to extract RNA.
3. The gene expression levels of the cytokines were determined, and the corresponding results are shown in FIG. 1.

As shown in FIG. 1, there is no significant difference in a relative expression of cytokines mRNA in HT29 cells between the control group and the HHP002 group, and the relative expression is very close, indicating that after acting on human colon cancer cells, the L. animalis strain HHP002 does not cause an up-regulation of cytokines expression in the human colon cancer cells.

However, there is a significant difference in the relative expression of cytokines mRNA in HT29 cells between the control group and the LPS group, indicating that LPS could cause an up-regulation of various pro-inflammatory cytokines in human colon cancer cells.

Several cytokines shown in FIG. 1 are closely related to the occurrence and development of tumors.

The IL-1β has been proved to promote a growth and transplantation of various tumors, and also cause fever and cachexia.

The IL-6 can promote tumor angiogenesis, in addition, the IL-6 can also inhibit human immune function and regulate the expression of cyclin through STAT3 activation, making tumor cells proliferate faster.

The IL-8 can enhance migration and infiltration ability of tumor cells, and also mediate the formation of drug resistance of tumor cells through PI3K/AKT/mTOR pathway. In addition, the IL-8 can also inhibit an activity of cytotoxic T cells and induce other immune cells to transform into tumor-related cells. Therefore, the serum level of L-8 in clinics is often negatively correlated with a therapeutic effect of tumor patients.

The IL-17 can act on fibroblasts, endothelial cells and tumor cells in tumor microenvironment, and induce them to produce more pro-inflammatory cytokines such as TNF-α, IL-6 and IL-1β. These cytokines can aggravate the inflammatory reaction and promote more inflammatory cells to infiltrate tumor tissue.

The TNF-α can induce tumor recurrence and metastasis of tumor cells.

Compared the LPS group with the LPS+HHP002 alone, the relative expressions of mRNA of IL-1β, IL-6, IL-8 and TNF-α in HT29 cells in the LPS+HHP002 group are significantly lower than the relative expressions of mRNA in the LPS group, while the relative expressions of mRNA of IL-1ß and TNF-α in LPS+HHP002 group are significantly different from the relative expressions of mRNA in LPS group. It shows that the L. animalis strain HHP002 can effectively inhibit the expression of pro-inflammatory cytokines in human colon cancer cells, thus inhibiting the further development of the tumor. When combined with other drugs, it is expected to achieve better curative effect.

In addition, the L. animalis strain HHP002 can also be used to prepare prophylactics, such as preventing the occurrence and recurrence of human colon cancer, which can be in the form of probiotic formulation.

### Embodiment 2, Acid Resistance and Choline Resistance Experiments

The pH value of human gastric juice varies from 1.5 to 4.5. If a strain can't survive in a low pH environment, the intake mode of the strain will be affected. The human large intestine contains bile salts with a concentration of between 0.3% and 0.5%. The existence of bile salts can inhibit the growth of some microorganisms. Therefore, a growth and survival in a presence of low pH value and bile salts are considered to be the most ideal characteristics of probiotic strains in the future.

The steps of the acid resistance experiments are as follows.

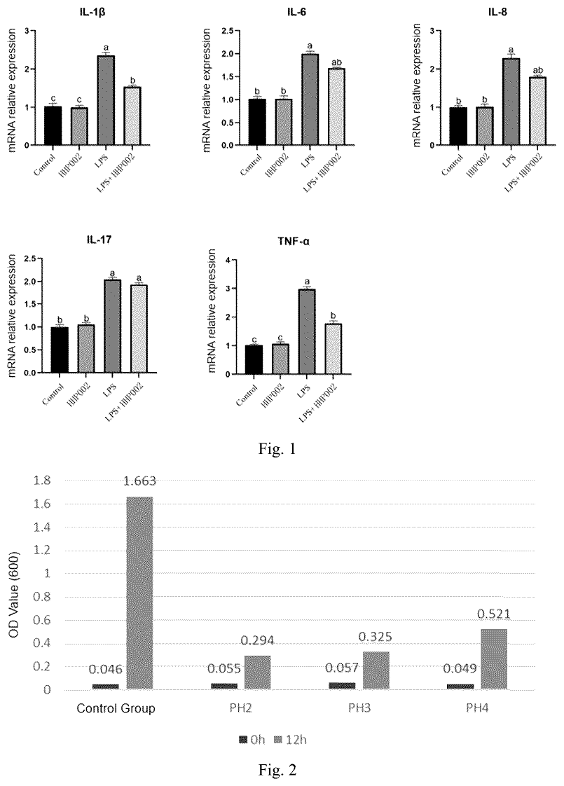

The pH value of the culture solution was adjusted to 2.0, 3.0, and 4.0, respectively, with 1 M of HCl, then the L. animalis strain HHP002 was inoculated and cultured at 37° C. for 12 hours. Compared with the untreated control group, the OD values at 0 hour and 12 hour respectively were measured to determine acid resistance, and the corresponding test results are shown in FIG. 2.

As shown in FIG. 2, the L. animalis strain HHP002 has good acid resistance and can still grow at low pH.

The steps of the choline resistance experiments are as follows.

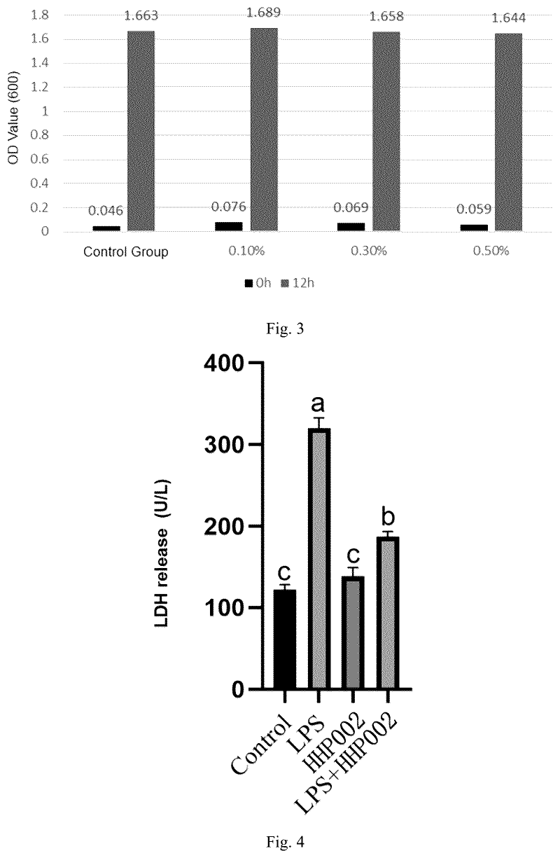

Firstly, 0.1%, 0.3% and 0.5% (w/v) of bovine choline were added to a culture solution, and the bovine choline was completely dissolved by shaking. Then, the L. animalis strain HHP002 was inoculated and cultured at 37° C. for 12 hours. Compared with the untreated control group, the OD values at 0 hour and 12 hour were measured respectively to determine its choline resistance. The corresponding test results are shown in FIG. 3.

As shown in FIG. 3, 0.1%, 0.3% and 0.5% (w/v) of bovine choline have no effect on the growth of the L. animalis strain HHP002, indicating that the L. animalis strain HHP002 has an ability to survive in human intestines.

### Embodiment 4, Cytotoxicity Test of Lactate Dehydrogenase (LDH)

The steps are as follows.

Experimental subjects were human colon cancer cell HT29. The experiment was divided into five groups, namely:
Cell culture medium group;
Control cell group treated at 37° C.: HT29 cells were cultured without other treatment;
L. animalis strain HHP002 group: when a confluence of the HT29 cells reached 80%, the HT29 cells were put into the cell culture medium containing L. animalis strain HHP002 (a concentration of the L. animalis strain HHP002 was 1×10⁷ CFU/mL) and co-cultured together for 6 hours;
LPS group: when a confluence of the HT29 cells reached 80%, 10 μg/mL of LPS was added and treated for 12 hours;
LPS+L. animalis strain HHP002 group: when a confluent of the HT29 cells reached 80%, the HT29 cells were first put into a cell culture medium containing the L. animalis strain HHP002 (a concentration of the L. animalis strain was 1×10⁷ CFU/mL) for 6 hours, and then 10 μg/mL of LPS was added treating for 12 hours;
After treatments, the LDH detection solution was added, mixed well, incubated at room temperature (about 25° C.) in the dark for 30 minutes, and then an absorbance was measured the at 490 nm;
The formula for calculating cytotoxicity or LDH enzyme activity is:

Cytotoxicity or LDH enzyme activity=(absorbance of treated sample-absorbance of sample control hole)/(absorbance of cell maximum enzyme activity-absorbance of sample control hole)×100.

FIG. 4 is the cytotoxicity test results of the L. animalis strain HHP002. As shown in FIG. 4, there is no significant difference in LDH release value between the L. animalis strain HHP002 group and the control cell group, indicating that the L. animalis strain HHP002 is not toxic to cells;

However, a LDH release value of the LPS group is significantly different from a LDH release value of the control group. After LPS treatment, the LDH release value of the HT29 cells is significantly increased, indicating that LPS cab damage HT29 cells.

A LDH release value of the LPS+L. animalis strain HHP002 group is significantly less than a LDH release value of the LPS group, indicating that the HT29 cells could obviously reduce the damage from LPS when the HT29 cells are incubated with the L. animalis strain HHP002 in advance. After cells are damaged, pro-inflammatory cytokines can be released. For patients with tumor recovery, an over-expression of pro-inflammatory cytokines may cause tumor recurrence. Therefore, the L. animalis strain HHP002 can be used as a probiotic formulation or used to prepare pharmaceutical composition for prevention to reduce the damage to intestinal cells, thus reducing the occurrence of inflammation and tumor recurrence.

It should be understood that, those skilled in the art can make improvements or transformations according to the above descriptions and schemes, and all these improvements and transformations should belong to the protection scope of the appended claims of the present disclosure.

What is claimed is:

1. A Ligilactobacillus animalis strain, wherein the L. animalis strain has been deposited in Guangdong Microbial Culture Collection Center on Oct. 13, 2022 with deposit number GDMCC NO.62867.

2. A use of the L. animalis strain according to claim 1 in a preparation of a pharmaceutical composition for inhibiting an expression of pro-inflammatory cytokines in human colon cancer.

3. The use according to claim 2, wherein the pro-inflammatory cytokines is one or more of IL-1B, IL-6, IL-8, and TNF-α.

4. A use of the L. animalis strain according to claim 1 in a preparation of a pharmaceutical composition for treatment of human colon cancer, a pharmaceutical composition for prevention of human colon cancer, and a probiotic formulation.
